(19) United States
(12) Patent Application Publication
(10) Pub. No.: US 2016/0121065 A1
(43) Pub. Date: May 5, 2016

(54) BREATHING ASSISTANCE APPARATUS HAVING CONTROLLABLE GASBAG AND METHOD FOR CONTROLLING THE GASBAG

(71) Applicant: DELTA ELECTRONICS, INC., Taoyuan County (TW)

(72) Inventors: Kuo-Liang LEE, Taoyuan County (TW); I-Chen MA, Taoyuan County (TW); Jung-Yu LIN, Taoyuan County (TW); Sheng-Wen PAI, Taoyuan County (TW)

(21) Appl. No.: 14/722,587

(22) Filed: May 27, 2015

(30) Foreign Application Priority Data
Oct. 31, 2014 (TW) 103137748

(51) Int. Cl. A61M 16/00; A61M 16/20; A61M 16/06

(52) U.S. Cl. CPC A61M 16/0078; A61M 16/0003; A61M 16/06; A61M 16/0066; A61M 16/20; A61M 2016/0027; A61M 2016/003; A61M 2205/3334; A61M 2205/50; A61M 2205/15

(57) ABSTRACT

A breathing assistance apparatus comprises a gas supply device, a mask prevent leakage control box (MPLCB), and a mask. The mask is used to cover user's face, and the mask has a gasbag at one side in contact with the user. The gas supply device connects to a gas inlet of the mask through a tunnel, and outputs treatment gas to the user stably. The MPLCB connects to the mask through a windpipe. The MPLCB pressurizes the gasbag automatically when a leakage is detected in the mask, and the gasbag is then inflated to touch the user's face. Therefore, the inside of the mask can remain airtight, so as to prevent the treatment gas in the mask from deflating out of the mask continually.

### BACKGROUND OF THE INVENTION

1. Field of the Invention

The invention relates to a breathing assistance apparatus, and in particularly to a breathing assistance apparatus capable of controlling inflating degree of a gasbag arranged on a mask thereof, and a method for controlling the gasbag.

2. Description of the Prior Art

To solve patient's respiration disorders, there are many kinds of assistance apparatus in the market used to assist patients to respire regularly. Generally speaking, these assistance apparatuses usually comprise a mask, and a gas supply device connected to the mask through a tunnel.

When using the assistance apparatus mentioned above, a user needs to wear the mask at a respiratory part (usually comprises nose and mouth), and the assistance apparatus provides an appropriate treatment gas (e.g., oxygen or other gases) by the gas supply device.

In particularly, the gas supply device mainly outputs the treatment gas with a certain pressure and amout of flow in accordance with doctor's instructions. The treatment gas is outputted to the mask through the tunnel for being absorbed by the user.

However, if the user wants to absorb the treatment gas outputted from the gas supply device normally, the user needs to wear the mask correctly to ensure an entire covering of the mask on user's face. If the user does not wear the mask well, or if the mask is moved during user's sleep, it may cause the mask to detach from user's face and no longer tight with user's face, so as to lead a leakage occurs.

If the leakage occurs, the pressure of the treatment gas is lower, the control of the pressure is unstable, and the treatment effect is reduced. Moreover, the user may feel uncomfortable because of the noise and the pressure generated by the leakage.

In the related art, the user needs to adjust the wearing position of the mask manually when the leakage occurs, so as to lead the mask to re-cover user's face entirely again. However, if the user is incapacitated or in sleep, and without any person around to provide an assistance, then the user cannot solve the leakage related problem.

### SUMMARY OF THE INVENTION

The object of the present invention is to provide a breathing assistance apparatus having controllable gasbag and a method for controlling the gasbag, the leakage related problem can be solved by controlling the gasbag arranged on a mask thereof to inflate.

The other object of the present invention is to provide the breathing assistance apparatus having controllable gasbag and the method for controlling the gasbag, the inflating degree of the gasbag of the mask can be adjusted to reduce the oppression of a user when the user wears the mask of the breathing assistance apparatus.

The breathing assistance apparatus of the present invention comprises a gas supply device, a mask prevent leakage control box (MPLCB), and a mask. The mask is used to cover user's face, and the mask has a gasbag at one side in contact with the user. The gas supply device connects to a gas inlet of the mask through a tunnel, and outputs treatment gas to the user stably. The MPLCB connects to the mask through a windpipe. The MPLCB pressurizes the gasbag automatically when a leakage is detected in the mask, and the gasbag is then inflated to touch the user's face. Therefore, the inside of the mask can remain airtight, so as to prevent the treatment gas in the mask from deflating out of the mask continually.

Comparing with prior art, the present invention can pressurize the gasbag to inflate to touch user's face tightly when the user does not wear the mask correctly and cause the leakage of the treatment gas in the mask. Therefore, there is no need to adjust the mask the user wore manually to lead the mask to re-cover with the user's face, and prevent the following leakage of the treatment gas in the mask.

Besides, if the mask is too tight to the user's face, the inflating degree of the gasbag can be further adjusted to reduce the oppression of the user when the user wears the mask. Therefore, the comfort of wearing the mask for a long term will be increased.

### BRIEF DESCRIPTION OF THE DRAWINGS

FIG. 1 is a block diagram of a first embodiment according to the present invention.

FIG. 2 is a diagram showing a mask of a first embodiment according to the present invention.

FIG. 3 is a flowchart for controlling a gas supply device of a first embodiment according to the present invention.

FIG. 4 is a flowchart for initially controlling a mask prevent leakage control box of a first embodiment according to the present invention.

FIG. 5 is a flowchart for preventing leakage of a first embodiment according to the present invention.

FIG. 6 is a flowchart for preventing leakage of a second embodiment according to the present invention.

FIG. 7 is a flowchart for preventing leakage of a third embodiment according to the present invention.

FIG. 8 is a block diagram of a second embodiment according to the present invention.

### DETAILED DESCRIPTION OF THE INVENTION

In cooperation with the attached drawings, the technical contents and detailed description of the present invention are described thereinafter according to a preferable embodiment, being not used to limit its executing scope. Any equivalent variation and modification made according to appended claims is all covered by the claims claimed by the present invention.

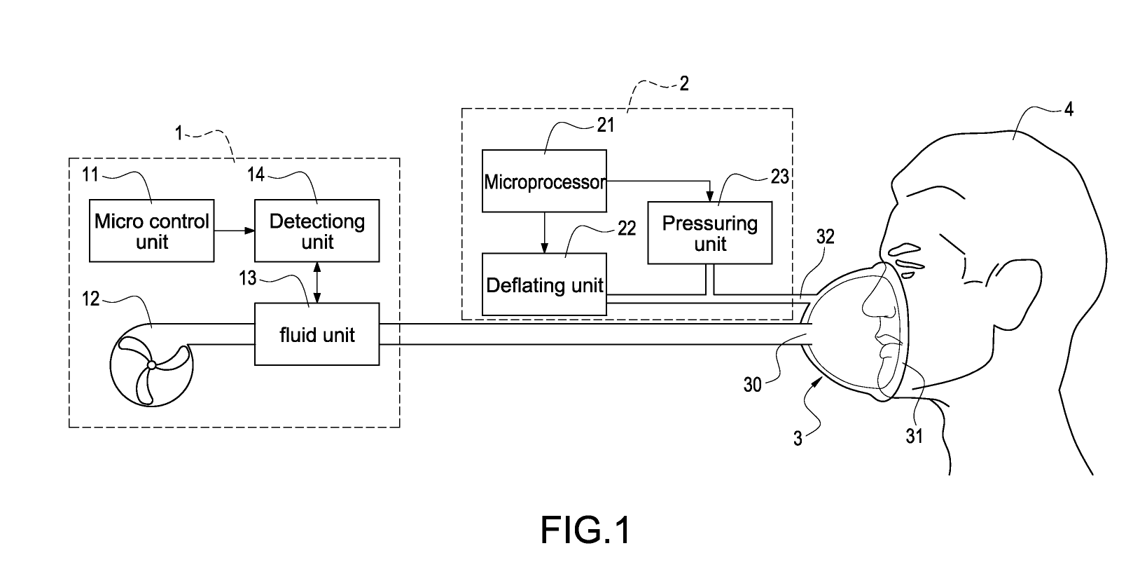

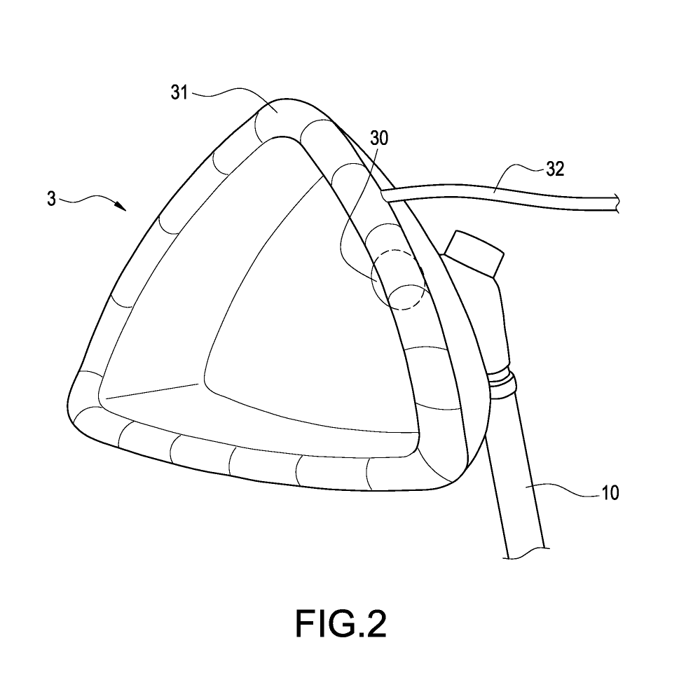

FIG. 1 is a block diagram of a first embodiment according to the present invention. FIG. 2 is a diagram showing a mask of a first embodiment according to the present invention. The present invention discloses a breathing assistance apparatus having a controllable gasbag. As shown in FIG. 1, the breathing assistance apparatus mainly comprises a gas supply device 1, a mask prevent leakage control box (MPLCB) 2 and a mask 3. The mask 3 is used to wear at a respiratory part (such as face) of a user 4, and to cover the face of the user 4. The mask 3 has a gasbag 31 arranged at one side in contact with the face of the user 4. The gas supply device 1 connects to the mask 3 through a tunnel 10. The gasbag 31 of the mask 3 connects to the MPLCB 2 through a windpipe 32, and is controllable by the MPLCB 2.

The gas supply device 1 comprises a micro control unit 11, a blower 12, a fluid unit 13 and a detection unit 14, wherein the micro control unit 11 is electrically connected with the blower 12 and the detection unit 14, the fluid unit 13 is connected with the blower 12 and the detection unit 14, and the fluid unit 13 is further connected to a gas inlet 30 of the mask 3 through the tunnel 10. In particularly, on end of the tunnel 10 is connected to the fluid unit 13, and other end of the tunnel 10 is connected to the gas inlet 30 of the mask 3. When the gas supply device 1 is activated, the blower 12 outputs wind to the fluid unit 13 for providing treatment gas from the fluid unit 13 to the mask 3 through the tunnel 10. In this embodiment, the fluid unit 13 can be, for example, a Venturi tube, a Pitot tube, or a honeycomb structure, not limited thereto.

Generally, the user 4 or a care giver can follow doctor's instructions to set an outputted pressure of the treatment gas, and a motor (not shown) of the blower 12 can run with a specific speed for the blower 12 to output a corresponding amount of wind, therefore, the fluid unit 13 can provide the treatment gas to the mask 3 in a corresponding pressure and a corresponding amoud of flow satisfied with the doctor's instructions. The detection unit 14 can be a pressure sensor or a flow sensor. The detection unit 14 is connected to the fluid unit 13, and it can determine if a leakage occurs at the mask 3 through the pressure and the amount of flow of the treatment gas. In this embodiment, the detection unit 14 determines if the leakage occurs or not by detecting the pressure of the treatment gas when the detection unit 14 is the pressure sensor. Else, the detection unit 14 determines if the leakage occurs or not by detecting the amount of flow of the treatment gas when the detection unit 14 is the flow sensor.

Besides, if the gas supply device 1 does not have the detection unit 14, it can still detect if the leakage occurs or not by determing the speed of the motor of the blower 12. In particularly, when the pressure of the treatment gas satisfies the doctor's instructions (i.e., matches to a target pressure), the gas supply device 1 determines if the speed of the motor of the blower 12 reaches a setting range or not, and determines the leakage occurs when the speed is higher than the setting range. If the speed of the motor of the blower 12 is lower than the setting range, the gas supply device 1 then determine that a tube bending problem probably occurs.

Under a regular condition, the user 4 usually absorbs stable amount of gas in a stable frequency, and the mask 3 usually has a standard deflating amount. For example, following the standard of ISO 17510, the deflating amount will be about 20 L/min for a 4 mm standard discouraged head with 10 cm H₂O. When the detection unit 14 detects the amount of flow of the fluid unit 13 is larger than the standard deflating amount (for example, the amount of flow of the fluid unit 13 reaches 100 L/min), then the gas supply device 1 can determine that the leakage occurs at the mask 3 side.

The micro control unit 11 controls the operating frequency of the blower 12, so as to control the outputted pressure and the amount of flow of the treatment gas. The micro control unit 11 further receives detection results from the detection unit 14, so as to determine if the leakage occurs or not at the mask 3 side. Furthermore, the micro control unit 11 controls the MPLCB 2 to solve the leakage related problem when the leakage occurs, or controls the blower 12 to increase pressure to compensate the pressure of the leakage.

The MPLCB 2 mainly comprises a microprocessor 21, a deflating unit 22 and a pressuring unit 23, wherein the microprocessor 21 is electrically connected to the deflating unit 22 and the pressuring unit 23, the deflating unit 22 and the pressuring unit 23 are connected to the windpipe 32 respectively, and are connected to the gasbag 31 of the mask 3 through the windpipe 32. In particuarly, one end of the windpipe 32 is connected to the gasbag 31, and other end of the windpipe 32 is connected to the pressuring unit 23 and the deflating unit 22 respectively. In this embodiment, the deflating unit 22 can be a solenoid valve or a standard discouraged valve, the pressuring unit 23 can be a pump or a turbine, the gasbag 31 can be a silicone gasbag or closing strips. However, the above description is just a specific embodiment, not intended to limit the scope of the present invention.

The microprocessor 21 of the MPLCB 2 and the micro control unit 11 of the gas supply device 1 can connect with each other for communication via a wireless transmission way or a physical cable (not shown) inside the tunnel 10. Further, the gas supply device 1 can provide power to the MPLCB 2 through the physical cable.

The deflating unit 22 is arranged to deflate gases inside the gasbag 31 regularly and quantitatively, so as to adjust the inflating degree of the gasbag 31. Therefore, the present invention can reduce the oppression of the user when the user wears the mask 3, and increase the comfort of wearing the mask for a long term.

If the user 4 does not wear the mask 3 correctly (e.g., the mask 3 does not cover user's face entirely), and the treatment gas in the mask 3 is deflated, the microprocessor 21 can receive a leakage notice from the micro control unit 11. The microprocessor 21 controls the pressuring unit 23 to pressurize the gasbag 31 after receiving the leakage notice, and the gasbag 31 is inflated to adjust and touch the user's face entirely, and leads the mask 3 to move to re-cover the user's face. Therefore, the leakage related problem is solved.

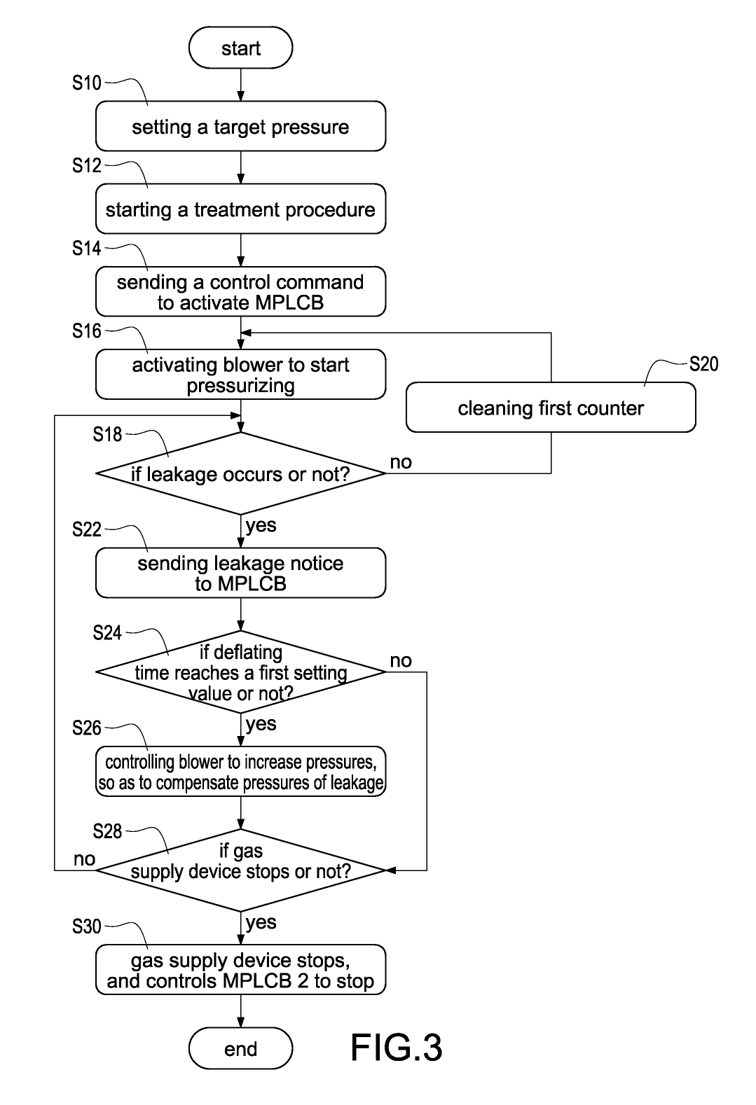

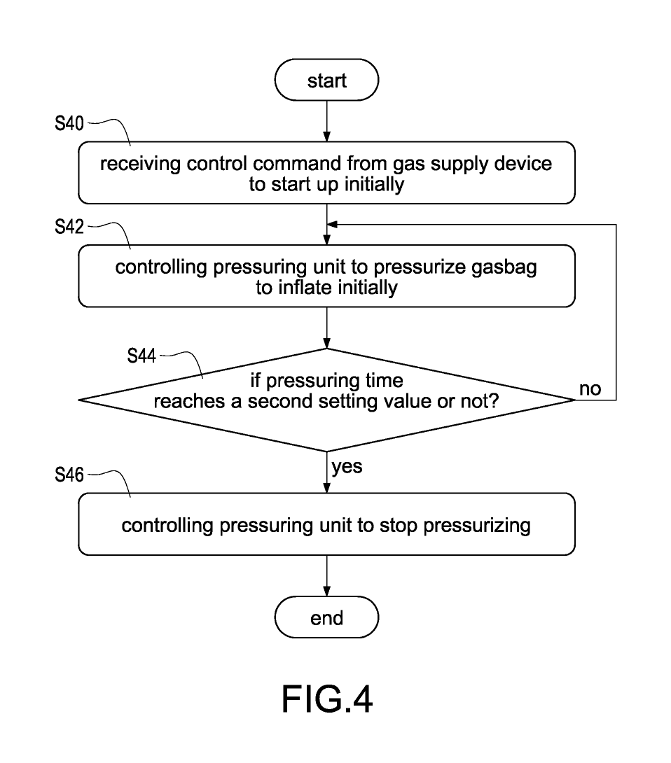

FIG. 3 is a flowchart for controlling a gas supply device of a first embodiment according to the present invention. FIG. 4 is a flowchart for initially controlling a mask prevent leakage control box of a first embodiment according to the present invention. The present invention also discloses a controlling method for the gasbag 31. Firstly, the gas supply device 1 is set with a target pressure in advance after being activated (step S10). In particularly, it is set with the speed of the motor of the blower 12 in accordance with the doctor's instructions, or set with the outputted pressure or the amount of flow of the treatment gas directly. After the step S10, the gas supply device 1 can start a treatment procedure (step S12). In the meanwhile, the user 4 needs to wear the mask 3 in a way that the mask 3 covers the user's face entirely.

After the treatment procedure starts, the gas supply device 1 sends a control command from the micro control unit 11 to the MPLCB 2 to activate the MPLCB 2 (step S14), and the MPLCB 2 is then triggered to initiate the gasbag 31 of the mask 3.

The micro control unit 11 then activates the blower 12 to start pressurizing after the step S14 (step S16). In this embodiment, the micro control unit 11 in the step S16 commands the blower 12 to increase the pressure, until the outputted pressure of the treatment gas can reach the target pressure. In this case, the detection unit 14 is used to detect if the outputted pressure of the treatment gas reaches the target pressure or not.

After the step S16, the gas supply device 1 keeps detecting the outputted pressure or the amount of flow of the treatment gas through the detection unit 14, and the micro control unit 11 keeps determining if the leakage occurs or not based on the detection result (step S18). In this embodiment, the micro control unit 11 determines if the leakage occurs or not by detecting if the outputted pressure or the amount of flow of the treatment gas is abnormal. For example, if the outputted pressure of the treatment gas is larger than the target pressure, or the outputted amout of flow of the treatment gas is larger than a target flow, the micro control unit 11 can determine that the leakage occurs. If there is no leakage occurs, then the gas supply device 1 cleans a first counter therein (step S20), wherein the first counter is used to count a deflating time (detailed described below).

If the leakage occurs, the micro control unit 11 sends the leakage notice to the MPLCB 2 (step S22), so as to command the MPLCB 2 to solve the leakage related problem via adjusting the inflating degree of the gasbag 31. After the step S22, the micro control unit 11 determines if the deflating time reaches a first setting value or not (step S24).

More specific, the micro control unit 11 uses the first counter to count the deflating time. If the deflating time reaches the first setting value, it shows that the MPLCB 2 and the gasbag 31 cannot solve the leakage related problem. In this situation, the micro control unit 11 controls the blower 12 to increase the pressures, so as to compensate pressures of the leakage (step S26). After the step S26, or after the micro control unit 11 determines the deflating time does not reach the first setting value, the micro control unit 11 further determines if the gas supply device 1 stops or not (step S28), i.e., if the treatment procedure ends or not. If not, the procedure is backs to the step S18, and the micro control unit 11 keeps determining if the leakage occurs or not. If yes, the gas supply device 1 stops and controls the MPLCB 2 to stop at the same time (step S30).

As shown in FIG. 4, after the step S14 in FIG. 3, the microprocessor 21 of the MPLCB 2 receives the control command sent from the mico control unit 11 of the gas supply device 1 (step S40), so as to start up initially. After the MPLCB 2 starts up, the microprocessor 21 controls the pressuring unit 23 to pressurize the gasbag 31 (step S42), so as to inflate the gasbag 31 initially. After the step S42, the microprocessor 21 determines if a pressuring time reaches a second setting value or not (step S44).

In particularly, the microprocessor 21 uses a second counter (not shown) in the microprocessor 21 to count the pressuring time of the pressuring unit 23. If the pressuring time reaches the second setting value, it shows that the gasbag 31 is in an appropriate inflating degree, so the microprocessor 21 controls the pressuring unit 23 to stop pressurizing (step S46). If the pressuring time does not reach the second setting value yet, the microprocessor 21 controls the pressuring unit 23 to keep pressurizing. The efficacy of the embodiment shown in FIG. 4 is that the gasbag 31 can be inflated to a certain degree in advance before the user 4 wears the mask 3, therefore, the user 4 can wear the mask 3 comfortably, and the contact part of the user 4 contacted with the mask 3 will not feel uncomfortable after wearing the mask 3.

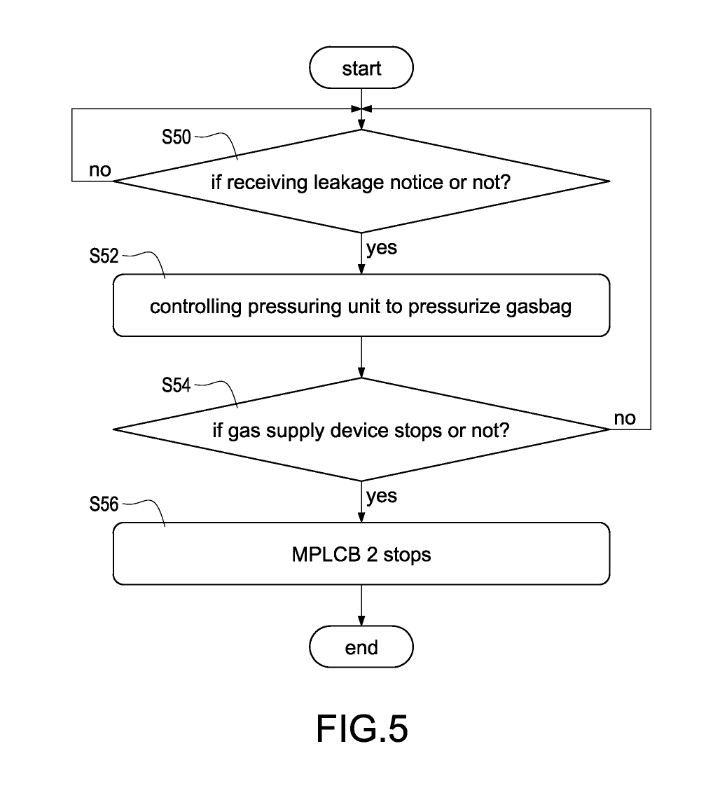

FIG. 5 is a flowchart for preventing leakage of a first embodiment according to the present invention. During the usage of the breathing assistance apparatus, the microprocessor 21 of the MPLCB 2 keeps determining if receiving the leakage notice from the gas supply device 1 or not (step S50). The leakage notice mentioned in this embodiment is the leakage notice sent from the micro control unit 11 in the above mentioned step S22 in FIG. 3.

Without receiving the leakage notice from the gas supply device 1, the MPLCB 2 does not pressurize the gasbag 31. On other hand, the microprocessor 21 of the MPLCB 2 controls the pressuring unit 23 to pressurize the gasbag 31 after receiving the leakage notice sent from the gas supply device 1 (step S52).

After the step S52, the microprocessor 21 determines if the gas supply device 1 stops or not (step S54). If the gas supply device 1 does not stop, the procedure is backs to the step S50, and the microprocessor 21 keeps determining if receiving the leakage notice from the gas supply device 1 or not. Therefore, the microprocessor can determine if the leakage related problem is solved or not by judging the leakage notice is received or not. On other hand, if the gas supply device 1 stops, then the MPLCB 2 stops, too (step S56). Else, the MPLCB 2 can also stop after receiving a stop command from the gas supply device 1.

In the above embodiment, the gasbag 31 is only pressurized to inflate when the leakage related problem of the mask 3 occurs. However, the microprocessor 21 can also adjust the inflating degress of the gasbag 31 for raising the comfort for the user 4 when the user wears the mask 3 in the following embodiment.

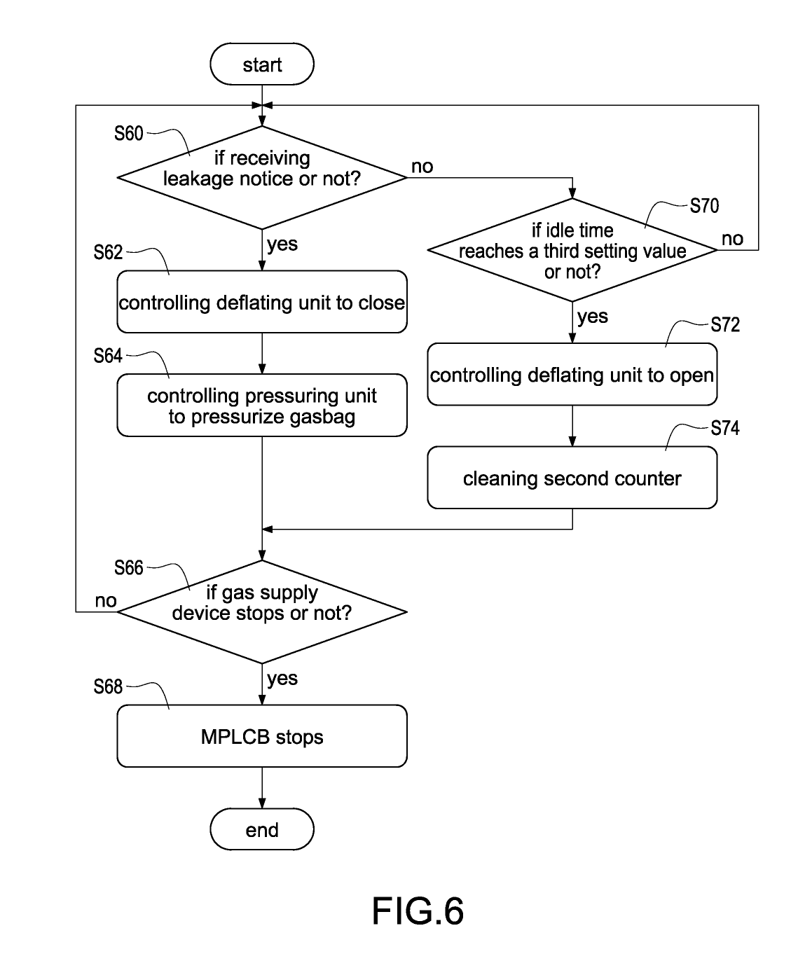

FIG. 6 is a flowchart for preventing leakage of a second embodiment according to the present invention. In the embodiment shown in FIG. 6, the MPLCB 2 comprises the deflating unit 22, and the deflating unit 22 in this embodiment is accomplished by a solenoid valve. In particularly, the solenoid valve is controlled by the microprocessor 21, and only open to deflate the gas in the gasbag 31 through the windpipe 32 when receiving an open instruction from the microprocessor 21. If the microprocessor 21 does not send the open instruction, the solenoid valve is always closed and prevent the outflow of the gas in the gasbag 31.

Firstly in this embodiment, the microprocessor 21 of the MPLCB 2 determines if receiving the leakage notice from the micro control unit 11 or not (step S60), if yes, the microprocessor 21 controls the deflating unit 22 to close (step S62), and controls the pressuring unit 23 to pressurize the gasbag 31 (step S64). The deflating unit 22 is used to deflate the gas in the gasbag 31, so the microprocessor 21 needs to close the deflating unit 22 in advance before executing the step S64, so as to control the pressuring unit 23 to pressurize the gasbag 31 effectively.

After the step S64, the microprocessor 21 determines if the gas supply device 1 stops or not (step S66). If the gas supply device 1 does not stop, the procedure is backs to the step S60, and the microprocessor 21 keeps determining if receiving the leakage notice from the gas supply device 1 or not. On other hand, if the gas supply device 1 stops, then the MPLCB 2 stops, too (step S68).

In this embodiment, if the MPLCB 2 does not receive the leakage notice from the micro control unit 11, it determines if an idle time reaches a third setting value or not (step S70). In particularly, the microprocessor 21 counts the idle time through the second counter during a period that the microprocessor 21 works normally and does not receive the leakage notice. If the idle time does not reach the third setting value, the microprocessor 21 keeps determining if receiving the leakage notice or not, and keeps determining if the idle time reaches the third setting value or not.

If the idle time reaches the third setting value, the microprocessor 21 controls the deflating unit 22 to open (step S72), so as to deflate the gas in the gasbag 31. In particularly, the microprocessor 21 mainly controls the deflating unit 22 to open for a while, and then controls the deflating unit 22 to close. Therefore, part of gas in the gasbag 31 is deflated appropriately to adjust the inflating degree of the gasbag 31. The execution of the step S72 can reduce the oppression of the user 4 when the user 4 wears the mask 3. After the step S72, the microprocessor 21 cleans the counting value of the second counter (step S74), so as to recount the idle time.

After the step S74, the microprocessor 21 determines if the gas supply device 1 stops or not, and determines if receiving the leakage notice or not again when the gas supply device 1 does not stop, and the microprocessor 21 stops when the gas supply device 1 stops.

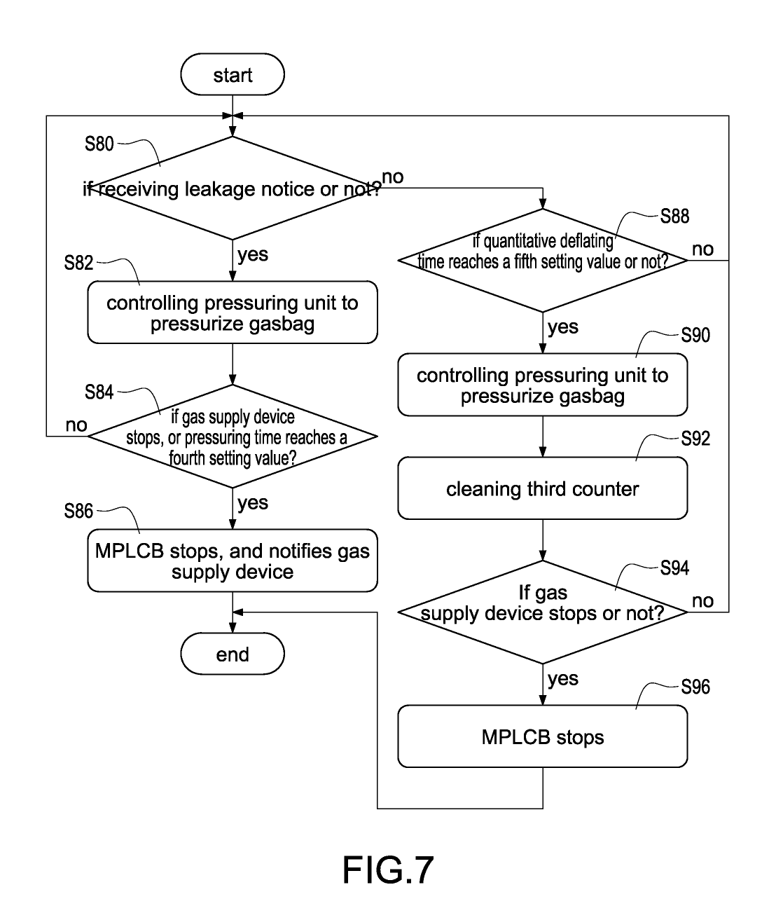

FIG. 7 is a flowchart for preventing leakage of a third embodiment according to the present invention. In this embodiment, the MPLCB 2 comprises the deflating unit 22, and the deflating unit 22 in this embodiment is accomplished by a quantitative discouraged valve. The difference between the quantitative discouraged valve and the solenoid valve is that the quantitative discouraged valve can deflate the gas in the pipe stably by itself (e.g., deflates 0.2 cc gases per minute), and is not controllable by the microprocessor 21.

Firstly in this embodiment, the microprocessor 21 of the MPLCB 2 determines if receiving the leakage notice from the micro control unit 11 or not (step S80), if yes, the microprocessor controls the pressuring unit 23 to pressurize the gasbag 31 (step S82). It should be mentioned that the deflating unit 22 in this embodiment is the quantitative discouraged valve, so the microprocessor 21 needs not to control the deflating unit 22 to close before executing the step S82.

After the step S82, the microprocessor 21 determines if the gas supply device 1 stops or not, or determines if a pressuring time reaches a fourth setting value or not (step S84). If the gas supply device 1 does not stop and the pressuring time does not reach the fourth setting value yet, the procedure is backs to the step S80, and the microprocessor 21 keeps determining if receiving the leakage notice from the gas supply device 1 or not. On other hand, if the gas supply device 1 stops, or the pressuring time reaches the fourth setting value, then the MPLCB 2 stops (step S86). In this embodiment, if the MPLCB 2 stops because the pressuring time reaches the fourth setting value, then the MPLCB 2 notifies the gas supply device 1 at the time the MPLCB 2 stops.

In this embodiment, the microprocessor 21 counts the pressuring time through the second counter during a period that the pressuring unit 23 pressurizes the gasbag 31. If the pressuring time does not reach the fourth setting value yet, the microprocessor 21 keeps determining if receiving the leakage notice or not, and keeps controlling the pressuring unit 23 to pressurize the gasbag 31 if the leakage notice is continually received. If the pressuring time reaches the fourth setting value, the microprocessor 21 controls the MPLCB 2 to stop.

If the MPLCB 2 does not receive the leakage notice from the micro control unit 11, it then determines if a quantitative deflating time reaches a fifth setting value or not (step S88). In particularly, the microprocessor 21 counts the quantitative deflating time through a third counter (not shown) during a period that the microprocessor 21 works normally and does not receive the leakage notice. If the quantitative deflating time does not reach the fifth setting value, the microprocessor 21 keeps determining if receiving the leakage notice or not, and keeps determining if the quantitative deflating time reaches the fifth setting value or not.

If the quantitative deflating time reaches the fifth setting value, the microprocessor 21 controls the pressuring unit 23 to pressurize the gasbag 31 (step S90), so as to increase the amount of the gas in the gasbag 31 and inflate the gasbag 31 with an appropriate size.

For example, a best amount of the gas in the gasbag 31 is 80 cc to 90 cc, and the deflating unit 22 can deflate 0.2 cc of gas quantitatively per minute. In this example, the microprocessor 21 controls the pressuring unit 23 to pressurize the gasbag 31 initially, makes the amount of the gas in the gasbag 31 reaches 90 cc, and counts a duty cycle through the third counter and the fifth setting value. When the deflating unit 22 deflates the gas in the gasbag 31 stably, and reduces the amount of the gas in the gasbag 31 to 80 cc after the duty cycle, the microprocessor 21 automatically controls the pressuring unit 23 to re-pressurize the gasbag 31, and makes the amount of the gas in the gasbag 31 recovering to 90 cc. Therefore, the amount of the gas in the gasbag 31 can be kept in an appropriate range through the cooperation of the deflating unit 22 and the pressuring unit 23, and the user 4 can have a best comfort when wearing the mask 3.

After the step S90, the microprocessor 21 cleans the counting amount of the third counter (step S92), so as to recount the quantitative deflating time. After the step S90, the microprocessor 21 determines if the gas supply device 1 stops or not (step S94). If the gas supply device 1 does not stop, the procedure is backs to the step S80, and the microprocessor 21 keeps determining if receiving the leakage notice from the gas supply device 1 or not, and keep determining if the quantitative deflating time reaches the fifth setting value or not. On other hand, if the gas supply device 1 stops, then the MPLCB 2 stops, too (step S96).

The breathing assistance apparatus in the present invention uses the above mentioned controlling method to control the gasbag 31, so as to solve the leakage related problem via pressurizing the gasbag 31 automatically when the leakage occurs at the mask 3 side. Besides, even there is no leakage occurs, the breathing assistance apparatus can still maintain the amount of the gas in the gasbag 31 in the appropriate range through deflating and pressurizing the gasbag 31 in certain times via the cooperation of the deflating unit 22 and the pressuring unit 23. Therefore, the user 4 will feel comfortable when wearing the mask 3.

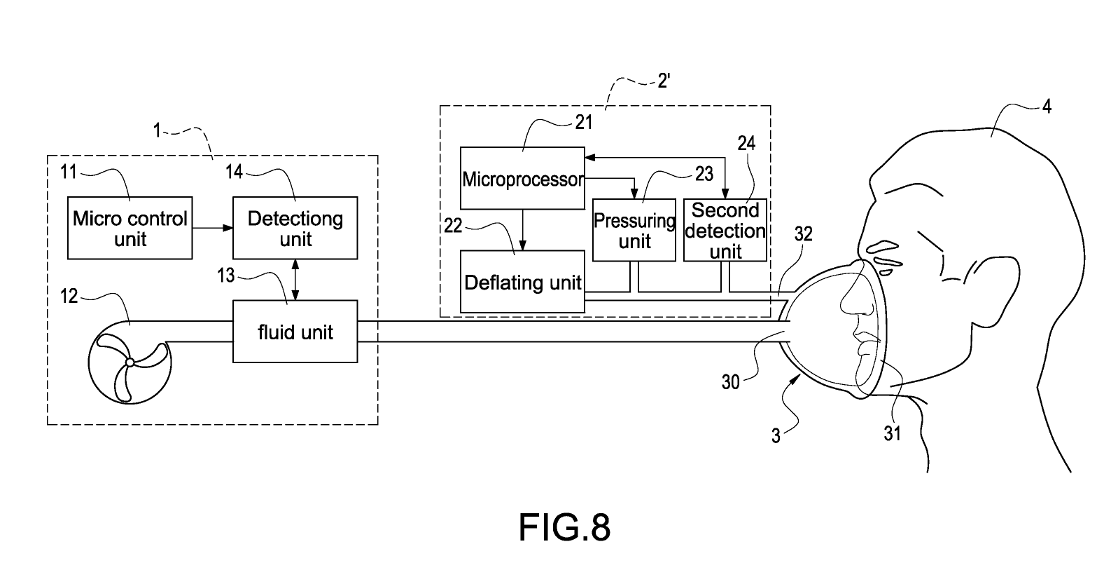

FIG. 8 is a block diagram of a second embodiment according to the present invention. FIG. 8 discloses other MPLCB 2′. The difference between the MPLCB 2′ and the above mentioned MPLCB 2 is the MPLCB 2′ further comprises a second detection unit 24, wherein the second detection unit 24 is electrically connected to the microprocessor 21, and further connected to the other end of the windpipe 32.

In this embodiment, the second detection unit 24 is a pressure sensor, and is used to detect the pressure status of the gasbag 31 through the windpipe 32. The efficacy of the second detection unit 24 is that the microprocessor 21 can adjust the deflating unit 22 (if the deflating unit 22 is the solenoid valve) and the pressuring unit 23 in anytime based on the detection result of the second detection unit 24, so as to keep the gasbag 31 in a best pressure status. Therefore, the user 4 can get the best comfort when wearing the mask 3.

As the skilled person will appreciate, various changes and modifications can be made to the described embodiment. It is intended to include all such variations, modifications and equivalents which fall within the scope of the present invention, as defined in the accompanying claims.

What is claimed is:

1. A breathing assistance apparatus, comprising:
a mask adapted for wearing at a respiratory part of a user, the mask having a gasbag at one side in contact with the user, and having a gas inlet thereon;
a gas supply device connected to the inlet through a tunnel, and the gas supply device outputting a treatment gas to the mask through the tunnel; and
a control box connected to the gasbag through a windpipe, and the control box controlling the gasbag to inflate when a leakage occurs at the mask side.

2. The breathing assistance apparatus according to claim 1, wherein the gasbag is a silicone gasbag or closing strips.

3. The breathing assistance apparatus according to claim 1, wherein the gas supply device comprises:
a blower;
a fluid unit connected to the blower and the tunnel, and the fluid unit provides the treatment gas to the mask through wind outputted by the blower;
a detection unit electrically connected to the fluid unit, and the detection unit detects outputted pressure or amount of flow of the treatment gas;
a micro control unit electrically connected to the detection unit and the blower, and the micro control unit determines if the leakage occurs at the mask side based on a detection result of the detection unit, and sends a leakage notic to the control box when the leakage occurs.

4. The breathing assistance apparatus according to claim 3, wherein the control box comprises:
a microprocessor receiving the leakage notice; and
a pressuring unit electrically connected to the microprocessor, and connected to the windpipe, wherein the pressuring unit pressurizes the gasbag according to a control by the microprocessor.

5. The breathing assistance apparatus according to claim 4, wherein the micro control unit of the gas supply device and the microprocessor of the control box connect with each other through a wireless way, or electrically connect with each other through a physical cable in the tunnel.

6. The breathing assistance apparatus according to claim 4, wherein the control box further comprises a deflating unit electrically connected to the microprocessor, and connected to the windpipe, and the deflating unit is used to deflate gas in the gasbag to adjust an inflating degree of the gasbag.

7. The breathing assistance apparatus according to claim 6, wherein the deflating unit is a solenoid valve or a quantitative discouraged valve

8. The breathing assistance apparatus according to claim 6, wherein the control bix further comprises a second detection unit electrically connected to the microprocessor, and connected to the windpipe, the second detection unit is used to detect pressure status of the gasbag, and the microprocessor controls the deflating unit and the pressuring unit based on a detection result of the second detection unit.

9. A method for controlling a gasbag applied to a breathing assistance apparatus, the breathing assistance apparatus comprising a gas supply device, a control box and a mask, the mask having a gasbag at one side, the gas supply device connected to an inlet of the mask through a tunnel and outputting a treatment gas to the mask, the control box connected to the gasbag through a windpipe, and the method comprising:
a) determining if receiving a leakage notice or not by the control box;
b) controlling a pressuring unit to pressurize the gasbag when receiving the leakage notice;
c) repeating the step a and the step b before the gas supply device stops; and
d) stopping working when the gas supply device stops.

10. The method according to the claim 9, wherein the control box comprises a deflating unit connected to the windpipe, and the deflating unit is a solenoid valve, the step b comprises following steps of:
b1) controlling the solenoid valve to close when receiving the leakage notice; and
b2) controlling the pressuring unit to pressurize the gasbag after the step b1, wherein the gasbag is inflated to touch user's face tightly and leads the mask to cover the user's face.

11. The method according to claim 10, wherein further comprises following steps of:
e) determining if an idle time reaches a first setting value or not when not receiving the leakage notice;
f) controlling the deflating unit to open to adjust an inflating degree of the gasbag if the idle time reaches the first setting value.

12. The method according to claim 9, wherein the control box comprises a deflating unit connected to the windpipe, and the deflating unit is a quantitative discouraged valve, wherein the control box detemines if the gas supply device stops or not, or determines a pressuring time of the pressuring unit reaches a second setting value or not in the step d, and the control box stops when the gas supply device stops or the pressuring time reaches the second setting value.

13. The method according to claim 12, wherein further comprises following steps of:
g) determining a quantitative deflating time reaches a third setting value or not when not receiving the leakage notice;
h) controlling the pressuring unit to pressurize the gasbag to adjust an inflating degree of the gasbag when the quantitative deflating time reaches the third setting value.

14. The method according to claim 9, wherein further comprises following steps before the step a:
a01) receiving a control command to start up initially at the control box; and
a02) controlling the pressuring unit to pressurize the gasbag to initially inflate according to the control command.

15. The method according to claim 14, wherein further comprises following steps of:
a03) determining if a pressuring time of the pressuring unit reaches a fourth setting value or not after the step a02;
a04) controlling the pressuring unit to stop pressurizing when the pressuring time reaches the fourth setting value.

16. The method according to claim 9, wherein further comprises following steps before the step a:
a05) setting a target pressure of the treatment gas at the gas supply device;
a06) starting a treatment procedure; and
a07) sending a control command to the control box to initially start up the control box after the step a06.

17. The method according to claim 16, wherein further comprises following steps of:
a08) determining if a leakage occurs at the mask side or not by detecting if an outputted pressure or amout of flow of the treatment gas is abnormal or not by the gas supply device;
a09) sending the leakage notice to the control box when the leakage occurs.

18. The method according to claim 17, wherein further comprises following steps of:
a10) determining if a deflating time reaches a fifth setting value or not by the gas supply device;
a11) controlling a blower to increase pressures to compensate pressures of the leakage when the deflating time reaches the fifth setting value.

19. A breathing assistance apparatus, comprising:
a mask having an gas inlet, and having a gasbag at one side in contact with a user;
a tunnel connected to the gas inlet at one end;
a blower;
a fluid unit connected to the blower and other end of the tunnel, outputting a treatment gas via wind outputted by the blower, and the treatment gas outputted to the mask through the tunnel and the gas inlet;
a detection unit electrically connected to the fluid unit, the detection unit detecting outputted pressure or amout of flow of the treatment gas;
a micro control unit electrically connected to the detection unit and the blower, the micro control unit sending a leakage notice when the outputted pressure or the amout of flow of the treatment gas is abnormal;
a windpipe connected to the gasbag at one end;
a microprocessor connected to the micro control unit, the micro processor receiving the leakage notice; and
a pressuring unit electrically connected to the micro processor, and connected to other end of the windpipe, the pressuring unit pressuring the gasbag to inflate after the microprocessor receiving the leakage notice.

20. The breathing assistance apparatus according to claim 19, wherein further comprises a deflating unit electrically connected to the microprocessor, and connected to the other end of the windpipe, and the deflating unit is used to deflate gas in the gasbag.
